(19) United States
(12) Patent Application Publication
(10) Pub. No.: US 2025/0090125 A1
(43) Pub. Date: Mar. 20, 2025

(54) PREDICTING THE DIAGNOSTIC CERTAINTY

(71) Applicant: Siemens Healthineers AG, Forchheim (DE)

(72) Inventor: Philipp Roser, Erlangen (DE)

(21) Appl. No.: 18/885,633

(22) Filed: Sep. 14, 2024

(30) Foreign Application Priority Data
Sep. 14, 2023 (EP) 23197481.7

(51) Int. Cl. A61B 6/00

(52) U.S. Cl. CPC A61B 6/54

(57) ABSTRACT

The aim is to provide better assistance to operators of X-ray imaging apparatuses in the use of X-ray images. To this end, a method is provided for operating an X-ray imaging apparatus by inputting to the X-ray imaging apparatus an object value that relates to an object to be X-rayed, and configuring an X-ray parameter of the X-ray imaging apparatus. A confidence value regarding a quality of the X-ray image to be acquired or reconstructed is determined using a model based on the object value and the X-ray parameter. The confidence value is provided for an operator or for the control of the X-ray imaging apparatus.

This application claims the benefit of European Patent Application No. EP23197481.7, filed on Sep. 14, 2023, which is hereby incorporated by reference in its entirety.

### BACKGROUND

The present embodiments relate to a method for operating an X-ray imaging apparatus by inputting to the X-ray imaging apparatus an object value that relates to an object to be X-rayed and configuring an X-ray parameter of the X-ray imaging apparatus. In addition, the present embodiments relate to an X-ray imaging apparatus.

Cone-beam computed tomography (CBCT) equipment and flat-panel detector angiography systems are often employed for interventional purposes (e.g., for intra-operative tracking of devices or for guiding such devices). The quality of the acquisitions is normally significantly lower than spiral computed tomography equipment, for example. It is becoming increasing desirable, however, to use cone-beam computed tomography equipment for voxel-based quantitative or at least qualitative diagnostic purposes. It may be the intention to use such systems to allow improved tissue or material differentiation (e.g., dual-energy material decomposition).

Diagnostic applications such as dual-energy diagnostics often rely heavily on the accuracy of the reconstructed Hounsfield units (HUs). The Hounsfield units are obtained from the Hounsfield scale that is used in computed tomography to describe attenuation of the X-rays in objects. Grayscale images may be represented by the Hounsfield units. Grayscale images are suitable for distinguishing between tissue types. This requires a certain HU fidelity, however. This HU fidelity does not exist in CBCT in general because of various effects (e.g., beam hardening, scatter, incomplete data, and so on).

Nonetheless, in many cases, the HU fidelity is sufficient to be able to apply diagnostic or quantitative algorithms with certainty. Before the actual CBCT scan, however, it is not known whether the required minimum HU fidelity may be achieved. Even after the acquisition, the assessment of the diagnostic certainty is normally based on the experience and intention of the radiologist.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the operator of an X-ray imaging apparatus is better assisted with a diagnosis.

The present embodiment hence provides a method for operating an X-ray imaging apparatus. The X-ray imaging apparatus may be, for example, the cone-beam computed tomography (CBCT) equipment mentioned in the introduction or a flat-panel detector angiography system. The method describes sub-acts for operating the X-ray imaging apparatus.

An object value relating to an object to be X-rayed is input to the X-ray imaging apparatus. The object to be X-rayed may be a patient or a technical subject, from whom or from which an X-ray image is to be obtained. The object value relates to, and describes, the object to be X-rayed. For example, the object value is the patient size, the patient weight, the material strength of a technical subject, the type of material of a technical subject, and suchlike. This object value is to be input to the X-ray imaging apparatus. A suitable interface may have to be provided for this purpose.

In a further act, an X-ray parameter of the X-ray imaging apparatus is configured. This configuration may also be performed at a suitable interface (e.g., of a control facility of the X-ray imaging apparatus). X-ray parameters may be, for example, the X-ray voltage, the acquisition time, and so on.

In a further act, a confidence value regarding a quality of an X-ray image to be acquired or reconstructed is determined by a model based on the object value and the X-ray parameter. The confidence value may also be referred to as a diagnostic trust value, because the confidence value specifies the certainty with which a certain quality of an X-ray image may be achieved. The quality may be assessed, for example, by the extent to which the quantity values of the X-ray image lie within a certain tolerance range. The confidence value is therefore used to predict the quality of the X-ray image to be acquired or reconstructed. The X-ray image may be a two-dimensional (2D) acquisition or a three-dimensional (3D) reconstruction of a plurality of 2D acquisitions.

The determining of the confidence value is performed by a model that may be varied by the at least one object value and the at least one X-ray parameter. If the object value relates to the anatomical region, for example, and specifies that the region to be X-rayed is an abdomen segment, then the model will, for example, lower the confidence value, because the X-ray scatter is relatively high in the abdomen and thus the diagnostic certainty falls. Conversely, the model will, if applicable, raise the confidence value if the X-ray voltage is increased, thereby increasing the contrast in some cases.

Finally, in a further act, the confidence value is provided for an operator or for the control of the X-ray imaging apparatus. For example, a total confidence value for a total X-ray image to be acquired or reconstructed may be provided as a number or color on a monitor. Alternatively, a confidence value distribution (e.g., as the “confidence value”) may also be presented (e.g., as a 2D or 3D graphic). An individual confidence value may be calculated for each pixel or voxel in this case. The presentation of the confidence value(s) on a monitor may make a diagnosis easier for the operator (e.g., because he is told that there is a 90% probability that it is liver tissue). Alternatively or additionally, the confidence value(s) may also be provided for controlling the X-ray imaging apparatus. A control facility may be needed for this purpose in order to store the confidence values and to read out these confidence values for the purpose of controlling the X-ray imaging apparatus. If applicable, the confidence values are provided to a suitable control input of the X-ray imaging apparatus.

It is thereby advantageously possible to provide a recommendation system or recommendation method that predicts the possible diagnostic certainty for a particular imaging protocol and/or an application. It is also advantageous that the present embodiments may be employed for dual-energy applications, although it is not limited thereto.

In an example embodiment, the confidence value relates to a reconstructed Hounsfield value. A 3D X-ray image may be reconstructed from a plurality of X-ray acquisitions, for example. Likewise, a virtual 3D image may be reconstructed from expected X-ray images that would be obtained from the at least one object and the at least one X-ray parameter by the model. The reconstructed 3D image would be a 3D dataset composed of Hounsfield values. The confidence value may then relate to the totality of the Hounsfield values, to just a portion of the Hounsfield values, or to just one single Hounsfield value.

In a further example embodiment, the confidence value is one of many confidence values, each of which is determined for a particular pixel or voxel of the X-ray image to be acquired or reconstructed. Thus, a confidence value is predicted for each pixel or voxel. What is known as a “heat map” may be created therefrom, which shows, for example, through color values, in which image regions there are low confidence values, indicating an expected poor image quality. An operator may then take suitable countermeasures (e.g., by configuring the X-ray imaging apparatus) if he is particularly interested in a specific image region.

According to a further example embodiment, a value range of Hounsfield values is assigned to a tissue type or object region, the confidence value is determined for the value range, and the confidence value is thereby assigned indirectly via the value range to the tissue type or object region. If, for example, liver tissue is meant to be detected, the Hounsfield values thereof then lie in a known value range for a patient of a certain size and certain weight, for example. The model may be used to determine the confidence value for this value range. Thus, a confidence value is assigned indirectly to the tissue type or object region. By the confidence value, the operator is given an indication of the certainty with which he may identify liver tissue for the patient (e.g., described by relevant object values) and for the selected configuration of the X-ray parameters of the X-ray imaging apparatus. A confidence value may be determined similarly for blood, for example, and the operator may alter the X-ray parameters of the X-ray imaging apparatus if the associated confidence value is too high or too low for him.

In one embodiment, the confidence value may also be determined for a region of the object or a region of the image (e.g., object region). For example, it is important to attain a high quality in a region around the spinal column. In this case, there should be a high confidence value for the selected region. The operator may influence this by the choice of X-ray parameters, for example. In the selected region, the confidence value may be determined, for example, by forming a mean value or forming a median value from a plurality of confidence values.

According to a further example embodiment, the confidence value is a value standardized for a particular slice of the X-ray image to be reconstructed. For example, it may be possible to achieve only different confidence values for different slices of the object. An inhomogeneity would thereby exist in relation to the slices. This inhomogeneity may be expressed by a homogeneity index. The homogeneity may be increased, for example, by using standardized or normalized confidence values for the individual slices. With regard to the homogeneity index, reference may be made to: Nowik P, Bujila R, Poludniowski G, Fransson A, Quality control of CT systems by automated monitoring of key performance indicators: a two-year study. J Appl Clin Med Phys. 2015 Jul. 8;16 (4): 254-265. doi: 10.1120/jacmp.v1614.5469. PMID: 26219012; PMCID: PMC5690007. This article defines the homogeneity key performance indicator (KPI) as the maximum difference in mean CT number (or Hounsfield unit) between the individual regions of interest (ROIs). The article also describes the uniformity as a further key index. The uniformity KPI is a test that evaluates the CT scanner performance in reconstructing a uniform image.

According to a further example embodiment, the providing of the confidence value is made in a map in which at each pixel location or voxel location is presented a corresponding confidence value. Thus, the heat map mentioned above is developed, which pinpoints each of the confidence values. The individual confidence values of the pixels or voxels are thus used not just to calculate, if applicable, a mean value, but the individual confidence values are also reproduced individually in two-dimensional or three-dimensional form in the corresponding representation. This may guide the operator more precisely through the configuration of the X-ray imaging apparatus.

In a further example embodiment, the determining of the confidence value takes into account a quality metric regarding a reconstructability of a voxel according to a detector trajectory of the X-ray imaging apparatus. For example, if it is barely possible to reconstruct a voxel for a particular CBCT trajectory or detector trajectory, then the confidence value may be lowered accordingly. Conversely, when the reconstructability is good, the confidence value for a voxel is raised accordingly. The reconstructability may be taken into account for each voxel individually.

In a further example embodiment, the object to be X-rayed is a patient, and the object value relates to at least one of the following values: size, weight, gender, water equivalent value, or anatomical region of the patient. Thus, the object value describes, for example, the patient. These patient values are used to determine the confidence values. One or more of these values may be used. For example, size and weight directly influence the X-ray result since the quality of the X-ray images depends on the given X-ray path. The same applies, for example, to the anatomical region of the patient, because more scattering is expected in the abdomen, for example, than in the head region.

In another example embodiment, the object value includes a 3D dataset that is obtained from the object, and an associated confidence value is determined for each voxel of the 3D dataset. For example, a 3D dataset is obtained from a patient pre-operatively or even intra-operatively, and is used for the prediction of further X-ray images to be acquired. For example, the 3D dataset may be used to establish a confidence value for each of its voxels using the model.

In a further embodiment, it may be provided that the determining of the confidence value is based on a statistical object model, which is parameterized by the at least one object value. For example, the statistical object model may be a statistical patient shape model or a statistical patient organ model, or suchlike. Using these models and, for example, size and weight figures, which are used to parameterize the models, the X-ray results may be predicted even more precisely. For example, the patient shape may be modeled very accurately by a patient shape model, if this is suitably parameterized by the size and/or weight of the patient. The same applies to organs of the patient, the form of which may be described very accurately by a statistical patient organ model parameterized by the size, weight, and/or gender of the patient.

In a further example embodiment, the X-ray parameter of the X-ray imaging apparatus relates to at least one of the following values: tube voltage, tube current, detector angle, or detector position. The X-ray imaging apparatus is configured by these X-ray parameters, which directly influence the X-ray result. For example, the tube voltage influences the energy distribution of the X-rays. The higher the tube voltage, the harder the radiation becomes, because the proportion of high-energy photons increases. The harder the X-rays, the more easily the X-rays penetrate matter. The material-dependent contrast may therefore be adjusted by the tube voltage. The intensity of the X-rays may be influenced by the tube current or heating current. Thus, these variables may be used to influence the brightness and contrast of the X-ray image and hence also the confidence value. Similarly, detector angle and detector position, and also angle and position of the radiation source, or their trajectories, may also have an influence on the X-ray images. Consequently, they may also affect reconstructions and the confidence values thereof.

In a further example embodiment, the determining of the confidence value is performed with the aid of a lookup table, a parameterizable function, or a machine learning algorithm. Simple value assignment is possible using the lookup table. With a parameterizable function, the assignment may be varied by the parameters. Maximum flexibility is achieved with a machine learning algorithm that is responsible for the assignment of the confidence value. For example, the machine learning may be performed by a neural network. Input variables may, for example, be the aforementioned object values and, if applicable, also a 3D dataset of the object. In addition, the X-ray parameters may also be input variables for the neural network. Output variables of the neural network or of the machine learning algorithm may accordingly be the confidence values.

According to a further example embodiment, a parameter of the X-ray imaging apparatus is configured automatically according to the confidence value provided. The provided confidence value is thereby used to control the X-ray imaging apparatus. The confidence value or the multiplicity of confidence values are used here to modify or configure automatically an X-ray parameter. For example, the tube voltage or a ratio of tube voltage and tube current may be modified automatically if the determined confidence value does not reach a preset magnitude. The X-ray imaging apparatus hence suggests a new parameter value that is configured for an acquisition to be performed. If then, for example, the operator accepts the suggestion, the X-ray acquisition may be carried out using the suggested parameter value.

The aforementioned object is also achieved according to the present embodiments by an X-ray imaging apparatus having: a first interface facility for inputting to the control facility an object value that relates to an object to be X-rayed and for configuring an X-ray parameter of the X-ray imaging apparatus; a computing facility configured to determine, using a model, based on the object value and the X-ray parameter, a confidence value regarding a quality of an X-ray image to be acquired; and a second interface facility for providing the confidence value for an operator or (for a control facility) for the control of the X-ray imaging apparatus.

The first interface facility for inputting the object value or patient value and for configuring the X-ray parameter may include a data interface, into which the relevant data may be input electronically. In addition, this first interface facility may also have configuration elements such as an adjusting wheel, a joystick, or a touchscreen. The computing facility for determining the confidence value may have at least one processor and at least one memory element. The second interface facility for providing the confidence value may have a monitor that is used to present visually the confidence value or a multiplicity of confidence values. If the confidence value(s) are used directly to control the X-ray imaging apparatus, the confidence values may also be provided by the second interface facility electronically for the X-ray imaging apparatus. For this purpose, the second interface facility may have, for example, a data input and, if applicable, a memory facility. The first and second interface facilities may also be integrated in one another.

In an example embodiment, the X-ray imaging apparatus is a cone-beam computed tomography device or an angiography device (e.g., flat-panel detector angiography device). Alternatively, however, the X-ray imaging apparatus may also be realized as spiral CT equipment or another X-ray device.

The advantages and possible variations mentioned above in connection with the method according to the present embodiments apply mutatis mutandis also to the X-ray imaging apparatus according to the present embodiments. Consequently, the features described in connection with the method may be regarded as functional features of the X-ray imaging apparatus.

As another example, a computer program that has commands that, when the commands are executed in a processor of the X-ray imaging apparatus of the type above, cause this apparatus to perform the above-described method is provided.

For cases of use or situations of use that may arise in the method and are not explicitly described here, it may be provided that, according to the method, an error message and/or a prompt to enter user feedback is output and/or a default setting and/or a predefined initial state is set.

Independent of the grammatical term usage, individuals with male, female, or other gender identities are included within the term.

### BRIEF DESCRIPTION OF THE DRAWINGS

The present embodiments are now explained in more detail with reference to the accompanying drawings, in which:

FIG. 1 shows a known C-arm angiography system having an industrial robot as a support apparatus; and

FIG. 2 shows a schematic method flow of an example embodiment.

### DETAILED DESCRIPTION

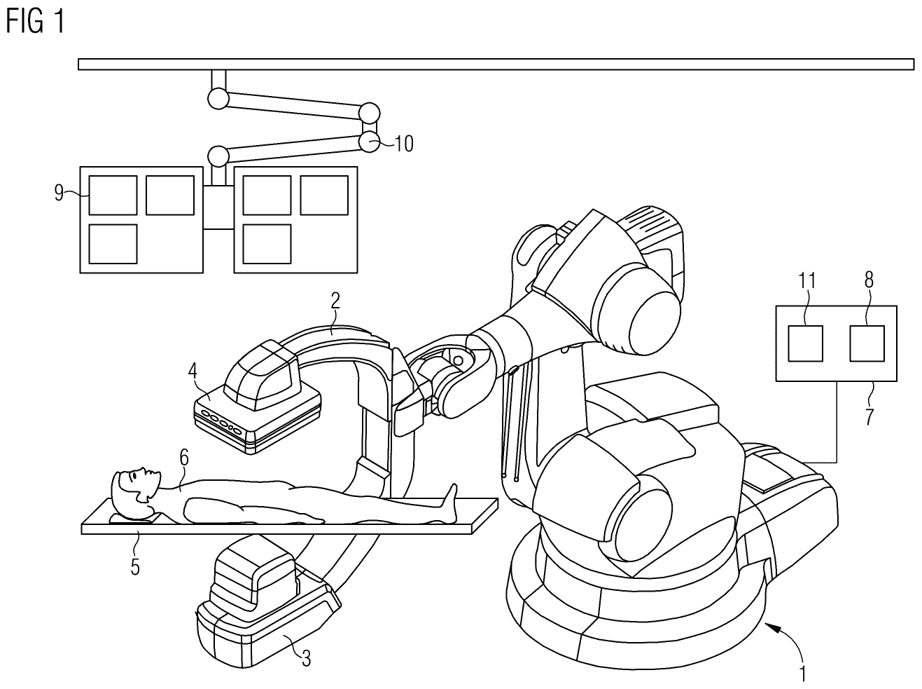

FIG. 1 shows by way of example a monoplane X-ray system having a C-arm 2 borne by a stand 1 in the form of a six-axis industrial or articulated robot. An X-ray radiation source (e.g., an X-ray source 3 having X-ray tube and collimator) and an X-ray image detector 4 are mounted on the ends of the C-arm 2 as an image acquisition unit. The realization of the X-ray diagnostic apparatus does not rely on the industrial robot. Standard C-arm devices may also be used.

In the beam path of the X-ray source 3, on a tabletop 5 of a patient positioning table, is a patient 6 to be examined or a technical subject as the object under examination. Connected to the X-ray diagnostic apparatus is a system control unit 7 having a computing apparatus 8 for image processing, which receives and processes the image signals from the X-ray image detector 4 (e.g., operator controls are not shown). The X-ray images may then be viewed on displays of a traffic-light monitor 9. The traffic-light monitor 9 may be borne by a support system 10 that may move longitudinally and is adjustable in angle, rotation, and height and has a boom and lowerable support arm. In the system control unit 7 is also provided a recommendation system 11 for determining a confidence value.

In the following example embodiment, a recommendation system that predicts the diagnostic certainty of a particular imaging protocol and/or application is provided. Although the present embodiments are explained using the example of dual-energy applications, the present embodiments are not limited to the use of dual energy.

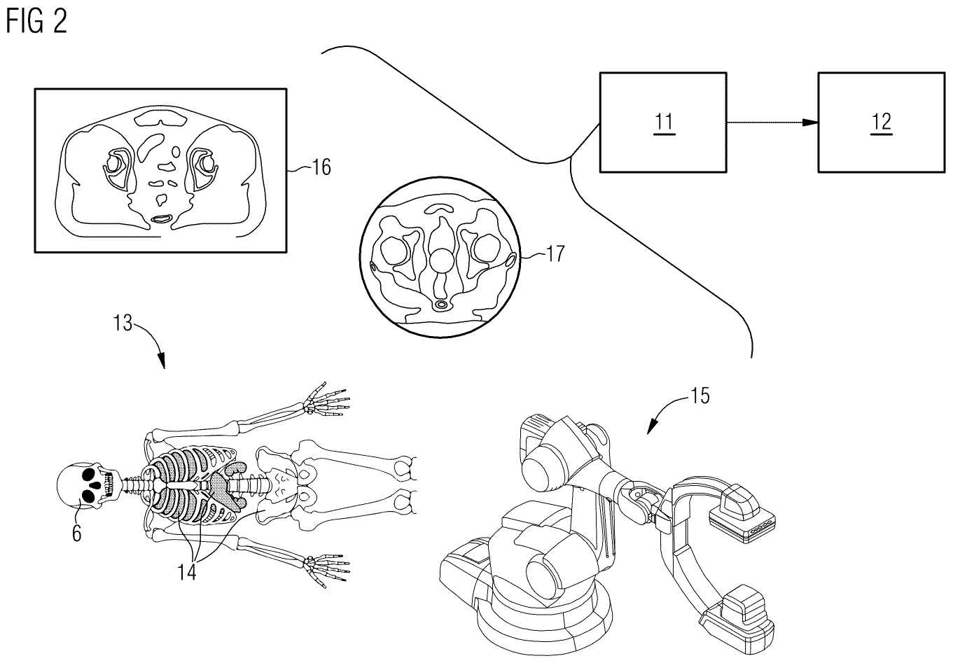

The present embodiments are based on the idea of using a recommendation system 11 to predict or output a diagnostic confidence value from at least one object value that describes an object to be X-rayed and at least one X-ray parameter that is used to configure an X-ray imaging apparatus. Various implementations of the diagnostic confidence value 12 of the input parameters (e.g., object value 13, X-ray parameter 15), and of the recommendation system 11 are presented below (cf. FIG. 2).

The diagnostic confidence value 12 constitutes a trust value that is intended to reflect the quality of an individual pixel, of an individual voxel, or of a pixel group or voxel group. The confidence value is calculated by a model and may have values in the range 0-1 or 0% to 100%, for example. 1 stands for, for example, the highest confidence (e.g., if a practically artifact-free CT scan is expected).

The confidence value 12, however, may also reflect what is known as an HU (Hounsfield) fidelity value. The HU fidelity value may specify, for example, the expected certainty or uncertainty of the reconstructed HU values. Optionally, the HU fidelity value may be specified according to a tissue type or for particular anatomical regions.

The confidence value 12 may also be a homogeneity index value, however, which represents a standardized value of the HU fidelity (see above).

In addition, the confidence value may be realized as a “heat map”. This provides that the confidence value is realized as a two-dimensional or three-dimensional value distribution. In this case, the aforementioned values are estimated per pixel or voxel for the reconstructable volume.

It is also possible, however, for the confidence value 12 to incorporate one or more requirements, for example, in addition to the HU fidelity value. For example, the recommendation system 11 (see FIG. 2) may use a completeness requirement for estimating the confidence value 12. It may hence be established for each pixel or voxel how well or completely it may be reconstructed for a particular CBCT trajectory. This reconstructability may be a supplementary feature for the estimate of the confidence value or equally may be a sole feature.

The recommendation system 11 requires a plurality of input parameters to estimate one or more confidence values or a confidence value distribution 12, as FIG. 2 illustrates. The recommendation system requires at least one object value 13 that describes the object (e.g., the patient 6). The object value may be, for example, the size, the weight, the gender, a water equivalent value, an anatomical region 14 of the patient 6. In principle, the object value may also be an object vector composed of a plurality of the aforementioned variables.

Examples of possible anatomical regions would be the skull, the lungs, the liver, the kidneys, the pelvis, etc. All of these regions have their own peculiarities with regard to the reconstruction, which may affect the confidence value.

In addition, the recommendation system 11 requires for determining the confidence value 12 at least one X-ray parameter 15, which is used to configure the X-ray imaging apparatus. The X-ray parameter may likewise be an X-ray vector composed of a plurality of parameters. An acquisition protocol of the X-ray imaging apparatus would be such a parameter vector. In FIG. 2, the C-arm device is used to symbolize the X-ray parameter(s) 15. As mentioned in the introduction, the X-ray parameter(s) relate to the tube voltage or the tube current, possible positions, and angular positions, or suchlike.

At least one statistical patient shape model or else a statistical organ model may be estimated based on the patient values or object values 13. The confidence value may be derived based on one or more of these models and, if applicable, the system parameters or X-ray parameters.

Combinations of the aforementioned object values or X-ray parameters may also be used to determine or estimate the confidence value 12.

A captured 2D or 3D dataset 16 of the object or patient 6 may also serve as the input parameters. Also, this captured 2D or 3D dataset 16 may be interpreted as an object value. The 2D or 3D dataset 16 provides information about the specific form or anatomy of the object. The 2D or 3D dataset 16 may be obtained pre-operatively or even intra-operatively, for example. During the operation, the CBCT acquisition 17, for example, may then be performed using the selected X-ray parameters. It can also be advantageous in some cases to capture the 2D or 3D dataset 16 even after the CBCT acquisition 17 (e.g., for reference purposes). These datasets may be obtained using a spiral CT system, for example, which exhibits a relatively high resolution and quality.

Alternatively or additionally, an earlier angiography or CBCT acquisition 17 may also serve as the input parameters for the recommendation system 11 for determining the confidence value 12. Such earlier acquisitions may serve as a reference value in order to determine the confidence value more reliably.

The recommendation system 11 may be based on different technologies. In the simplest case, a lookup table (LUT) is used in order to assign an output value (e.g., confidence value) to the input parameters. A lookup table of this type is inflexible, however. Greater flexibility with regard to assigning one or more confidence values to a set of input parameters may be obtained by a parameterizable function. The assignment rule may even be modified by parameters here. These parameters may be adapted either manually or automatically. Automatic adaptation may be used, for example, to spread the confidence values in order to use an entire desired range of values.

Even greater flexibility in the assignment of confidence values to the input parameters is achieved when using machine learning algorithms. Such algorithms may be based on gradient boosting, neural networks, convolutional neural networks, transformers, reinforcement learning, large language models (LLMs), and so on. Input variables to such an algorithm would be the input parameters, and the output variable would be the confidence value or the confidence value distribution.

In an embodiment, it may be provided that the severity of different artifacts is specified separately. For example, the scattered radiation in the abdominal cavity is more pronounced than in the skull region. In addition, recommendations may be specified for improving the determined confidence values. For example, it may be recommended to use collimators in order to reduce the scattered radiation.

In a further embodiment, the diagnostic confidence value may be estimated right at the beginning of an intervention (e.g., if it is known in advance that a particular CBCT application is necessary during the intervention).

The approaches according to the present embodiments provide the advantage that it may be possible to save an X-ray dose if the determined confidence value(s) cause an operator not to carry out the resultant CBCT, for example. In addition, the workflow (e.g., for administering a contrast agent) may be improved, because the transfer (e.g., to the CT) can be planned accordingly. A further advantage of the present embodiments is that a radiologist has to hand an objective model when planning the intra- or post-operative treatment based on a diagnostic (CB) CT.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for operating an X-ray imaging apparatus, the method comprising:
inputting to the X-ray imaging apparatus an object value relating to an object to be X-rayed;
configuring an X-ray parameter of the X-ray imaging apparatus;
determining, using a model, based on the object value and the X-ray parameter, a confidence value regarding a quality of an X-ray image to be acquired or reconstructed; and
providing the confidence value for an operator or for control of the X-ray imaging apparatus.

2. The method of claim 1, wherein the confidence value relates to a reconstructed Hounsfield value.

3. The method of claim 1, wherein the confidence value is one of a number of confidence values, each of the number of confidence values being determined for a particular pixel or voxel of the X-ray image to be acquired or reconstructed.

4. The method of claim 1, wherein a value range of Hounsfield values is assigned to a tissue type or object region, the confidence value is determined for the value range, and the confidence value is thereby assigned indirectly via the value range to the tissue type or object region.

5. The method of claim 1, wherein the confidence value is a value standardized for a particular slice of the X-ray image to be reconstructed.

6. The method of claim 1, wherein providing the confidence value comprises providing the confidence value in a map in which at each pixel location or voxel location a corresponding confidence value is presented.

7. The method of claim 1, wherein the determining of the confidence value takes into account a quality metric regarding a reconstructability of a voxel according to a detector trajectory of the X-ray imaging apparatus.

8. The method of claim 1, wherein the object to be X-rayed is a patient, and the object value relates to size, weight, gender, water equivalent value, anatomical region of the patient, or any combination thereof.

9. The method of claim 1, wherein the object value comprises a three-dimensional (3D) dataset that is obtained from the object, and
wherein the method further comprises determining an associated confidence value for each voxel of the 3D dataset, the associated confidence values comprising the confidence value.

10. The method of claim 1, wherein the determining of the confidence value is based on a statistical object model that is parameterized by the object value.

11. The method of claim 1, wherein the X-ray parameter of the X-ray imaging apparatus relates to tube voltage, tube current, detector angle, detector position, or any combination thereof.

12. The method of claim 1, wherein the determining of the confidence value is performed with the aid of a lookup table, a parameterizable function, or a machine learning algorithm.

13. The method of claim 1, wherein a parameter of the X-ray imaging apparatus is configured automatically according to the confidence value provided.

14. A control facility for an X-ray imaging apparatus, the control facility comprising:
a first interface facility for inputting to the control facility an object value that relates to an object to be X-rayed and for configuring an X-ray parameter of the X-ray imaging apparatus;
a computing facility configured to determine, using a model, based on the object value and the X-ray parameter, a confidence value regarding a quality of an X-ray image to be acquired; and
a second interface facility configured to provide the confidence value for an operator or for control of the X-ray imaging apparatus.

15. An X-ray imaging apparatus comprising:
an X-ray imaging unit; and
a control facility that is configured to control the X-ray imaging unit, the control facility comprising:
a first interface facility for inputting to the control facility an object value that relates to an object to be X-rayed and for configuring an X-ray parameter of the X-ray imaging apparatus;
a computing facility configured to determine, using a model, based on the object value and the X-ray parameter, a confidence value regarding a quality of an X-ray image to be acquired; and
a second interface facility configured to provide the confidence value for an operator or for control of the X-ray imaging apparatus.
